(19) United States
(12) Patent Application Publication
(10) Pub. No.: US 2026/0053560 A1
(43) Pub. Date: Feb. 26, 2026

(54) COOLED ANTENNAS FOR SPHERICAL ABLATION

(71) Applicant: Neuwave Medical, Inc., Madison, WI (US)

(72) Inventor: Matthew J. THIEL, Verona, WI (US)

(73) Assignee: Neuwave Medical, Inc., (US)

(21) Appl. No.: 18/810,315

(22) Filed: Aug. 20, 2024

(51) Int. Cl. A61B 18/18; A61B 18/00

(52) U.S. Cl. CPC A61B 18/1815; A61B 2018/00017; A61B 2018/1869; A61B 2018/1892

(57) ABSTRACT

An energy delivery device is disclosed including a cannula extendable through skin of a patient and an antenna extending from the cannula and operable to deliver energy to tissue of the patient. The antenna includes an outer conductor, a dielectric tube extending within the outer conductor, a conductor extending within the dielectric tube, and a coolant tube extending within the dielectric tube. Coolant is to be supplied through the coolant tube.

### BACKGROUND

Ablation is an important therapeutic strategy for treating certain tissues, such as benign and malignant tumors, cardiac arrhythmias, cardiac dysrhythmias, and tachycardia. Some ablation systems utilize radio frequency (RF) energy as the ablating energy source. However, RF energy has several limitations, including the rapid dissipation of energy in surface tissues resulting in shallow “burns” and failure to access deeper tumor or arrhythmic tissues. Another limitation of RF ablation systems is the tendency of eschar and clot formation on the energy emitting electrodes, which limits further deposition of electrical energy.

More recently, microwave energy is being used as the ablating energy source in ablation systems. Microwave energy is an effective energy source for heating biological tissues, and is used in applications such as cancer treatment and preheating of blood prior to infusions. One advantage of microwave energy over RF is the deeper penetration into tissue, insensitivity to charring, lack of necessity for grounding, more reliable energy deposition, faster tissue heating, and the capability to produce much larger thermal lesions than RF, which greatly simplifies the actual ablation procedures.

Conventional microwave ablation devices typically generate ablations that exhibit a generally ellipsoid or teardrop-shaped ablation zone. This is due to several factors related to the physics of the antennas of microwave ablation devices. A first factor is that the wavelength of microwave energy requires the antenna of the ablation device to be long and, thus, the application of energy occurs along the antenna length rather than from a point source. A second factor is that the dielectric properties of tissue change by more than an order of magnitude as the tissue temperature changes from body temperature to greater than 150° C., which is typically observed at the hottest point of a high-power ablation. The wide range of properties creates an inefficient, non-optimized environment for the antenna to operate. Both the antenna efficiency and the shape of the ablation degrades as the tissue desiccates and chars, creating back-heating along the length of the device. A third factor is that, in a coaxial fed antenna, current is often conducted backwards on the outer conductor surface and are absorbed by tissue surrounding the probe proximal to the antenna.

Physicians have voiced a desire for microwave ablation devices that can generate a spherical microwave ablation zone. For ablation with a single probe, a spherical zone is advantageous as it allows for the least amount of collateral damage for a given target lesion, allows for shallower placement without skin/body wall burns, and allows for more options in probe placement as the spherical zone can cover the same target tissue regardless of approach angle.

Accordingly, there is a need for improved systems and devices for generating a spherical microwave ablation zone with a microwave ablation device.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example energy delivery system that may be used in accordance with the principles of the present disclosure.

FIG. 2 is a schematic diagram of an example energy delivery device with an antenna for generating a spherical ablation zone, according to at least one aspect of the present disclosure.

FIG. 3 is a detailed, cross-sectional view energy of the antenna of FIG. 2, according to at least one aspect of the present disclosure.

FIG. 4 is a cross-sectional view of the antenna of FIG. 2, according to at least one aspect of the present disclosure.

### DETAILED DESCRIPTION

The present disclosure is related to systems and methods for delivering energy to tissue for ablation operation and, more particularly, to systems and methods for controlling the shape of an ablation zone generated by an energy delivery device.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof.

More specifically, the present disclosure is related to creating spherical microwave ablations using an antenna that is optimized for spherical field pattern, high power handling, and efficiency. The example antenna described herein incorporates a choke that prevents back heating, and also minimizes the change in dielectric properties in the immediate vicinity of the antenna so the antenna can operate efficiently and with the desired field pattern throughout the ablation process. Dielectric property management can be achieved by cooling the tissue in the active zone of the antenna to prevent charring, but in applications where carbon dioxide (CO₂) gas is used as a coolant, the CO₂ gas must be contained in a robust sealed chamber to prevent escape into the body of the patient. The structures and antennas described herein allow robust, safe CO₂ cooling of the active zone while incorporating an efficient choke.

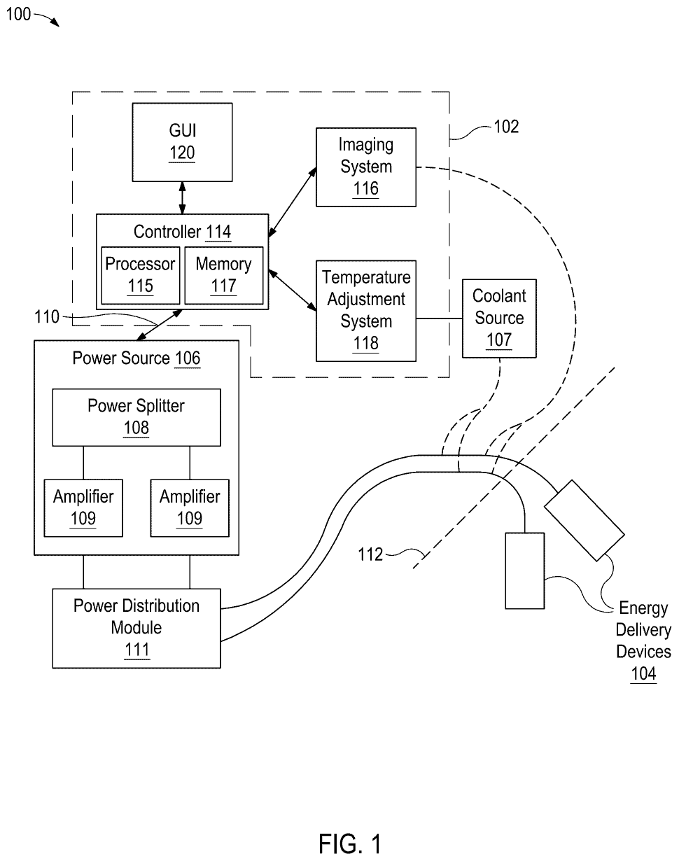

FIG. 1 is a block diagram of an example energy delivery system 100 that may incorporate the principles of the present disclosure. As illustrated, the energy delivery system 100 (hereafter “the system 100”) includes a control system 102 and one or more energy delivery devices or “ablation probes” 104 (two shown) designed to deliver (emit) energy to a target tissue region of a patient. While two ablation probes 104 are shown, the system 100 may include only one ablation probe 104 or more than two ablation probes 104 (e.g. three, four, or five ablation probes) An example ablation probe 104 that can be used with the energy delivery system 100 is described in U.S. Patent Application Publication No. 2021/0282851, entitled “ENERGY DELIVERY SYSTEMS AND USES THEREOF”, which published on Sep. 16, 2021, the disclosure of which is hereby incorporated by reference in its entirety.

The system 100 further includes a power source or generator 106 communicably coupled to the control system 102 and the ablation probes 104 to direct, control, and deliver (provide) electrical power thereto. In some applications, the power source 106 may include a power splitter 108 that receives power from an external power source (e.g. a wall outlet) and directs power to one or more amplifiers 109 (two shown), which amplify the voltage, current, or power from the power splitter 108 to an associated ablation probe 104. While two amplifiers 109 are shown, each associated with a corresponding ablation probe 104, the power source 106 may include less than two amplifiers (e.g. one amplifier) or more than two amplifiers (e.g. three, four, or five amplifiers, for example). Each amplifier 109 may be coupled to a corresponding ablation probe 104 via a power distribution module 111, which provides strain relief to the cabling extending from the amplifiers 109 to the ablation probes 104. The power distribution module 111 may be coupled to a structure in the operating room, such as a surgical bed, and may house connection hardware of the probes 104.

The components of the system 100 are connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

The control system 102 is configured to monitor, control, and provide feedback concerning operation of the system 100. As illustrated, the control system 102 includes at least a controller 114, an imaging system 116, and a temperature adjustment system 118. The control system 102 may further include a graphical user interface (GUI) 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region and accessible by an operator to operate and/or monitor the components of the system 100.

The controller 114 may include a processor 115 and a memory or memory device 117 comprising any storage media readable by the processor 115. The memory 117 may store software or software instructions executable by the processor 115 to carry out functions and operations of the system 100. Examples of the memory 117 include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 115. Examples of computer readable media include, but are not limited to, optical discs, magnetic disks, magnetic tape, solid-state media, and servers for streaming media over networks.

Based on instructions provided by the software, the controller 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The controller 114 interacts with the ablation probes 104 to raise or lower (e.g., tune) the amount of energy delivered to the tissue region. The controller 114 may also be configured to prime coolants for distribution into the ablation probes 104 such that the coolant is delivered at a desired temperature.

In some applications, the type of tissue being treated is inputted into the software for purposes of allowing the controller 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected ablation sizes may be based on tissue type. In such embodiments, the controller 114 may not control power delivery based on tissue type. In yet other embodiments, the controller 114 generates a chart or diagram based upon a particular type of tissue or tissue region displaying characteristics useful to a user of the system.

The controller 114 may allow a user to choose power, duration of treatment, different treatment algorithms for different tissue types, simultaneous application of power to multiple probes 104, coherent and incoherent phasing, etc. The controller 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

As discussed above, physicians desire a microwave ablation device that can generate a spherical microwave ablation zone. Despite this clear desire, typical microwave ablation devices generate an ellipsoid or teardrop-shaped microwave ablation zone due to several factors related to the physics of the antennas of microwave ablation devices.

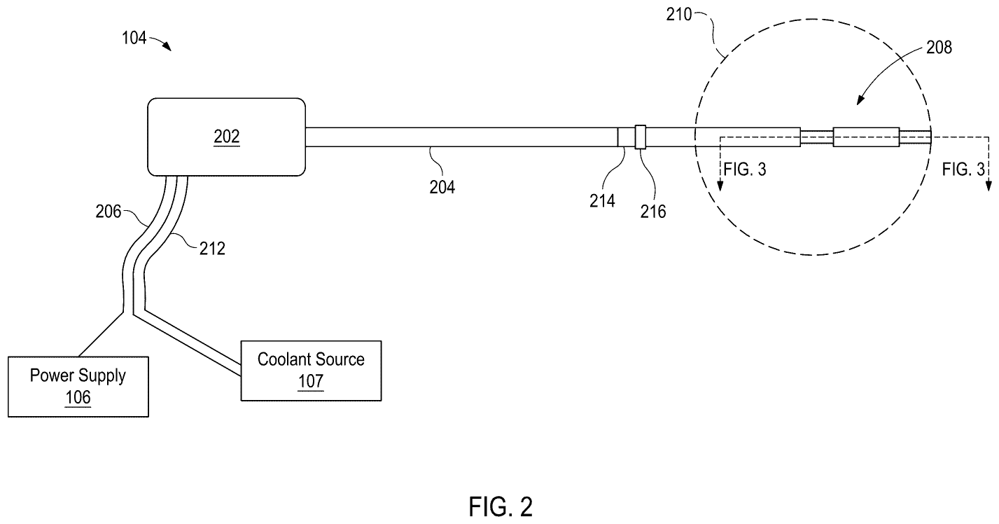

FIG. 2 is a schematic diagram of an example ablation probe 104 for generating a spherical ablation zone, according to at least one aspect of the present disclosure. The ablation probe 104, as will be described in more detail below, is designed to combat the factors that contribute to the ellipsoid or teardrop-shaped ablation zone in typical microwave ablation devices. As used herein, the term “spherical” as used in “spherical ablation zone” can refer to an ablation zone that exhibits a general shape of a sphere having substantially the same radius in all angular directions, but could also refer to a sphere-like structure that includes slight variations in radius resulting from differing properties of adjacent tissue. For instance, the ablation zone may described in terms of its aspect ratio, which is the ratio of its width or maximum diameter in the transverse plane to its length along the longitudinal axis. A spherical ablation zone may be defined as having an aspect ratio between about 0.9 and about 1.0. The term “spherical” is contrasted against the terms “ellipse” or “ellipsoid,” as in an “ellipsoid ablation zone,” which exhibits a rounded shape having a length that is greater than its height, such as a length that is at least 1.5 times greater than a height, or has an aspect ratio less than about 0.9, such as about 0.6 to about 0.75, for example.

The ablation probe 104 may be configured to deliver (emit) energy (e.g., microwave energy, radiofrequency energy, radiation energy) to a target tissue region. As illustrated, the ablation probe 104 includes a handle housing 202 and an elongate shaft or probe cannula 204 extending distally from the handle 202. In some embodiments, the probe cannula 204 may be rigid (e.g., for percutaneous use), but could alternatively be flexible (e.g., for navigating through a bronchoscope). In some embodiments, the probe 104 may not include the handle housing 202.

A cable or cable assembly 206 may be operatively coupled to the handle 202 and configured to convey electrical power thereto. The cable assembly 206 may extend from the power supply 106 (FIG. 1), for example, and may provide the power sufficient to operate the ablation probe 104. An antenna 208 is provided at the distal end of the probe cannula 204 and receives electrical power from the cable assembly 206 to emit energy (e.g., microwave energy) to a target tissue region and thereby generate a spherical, or substantially spherical, ablation zone 210, as discussed in more detail below. In at least one application, the ablation probe 104 includes two or more separate antennae 208 attached to the same or different power supplies.

In some applications, a cooling tube 212 is operatively coupled and configured to convey a cooling fluid or “coolant” to the handle 202. The handle 202 may be configured to control conveyance of the cooling fluid into and out of the probe cannula 204 to help regulate a temperature of the antenna 208 and the ablation zone 210. In other embodiments, the control system 102 (FIG. 1) may be configured to control conveyance of the cooling fluid into and out of the probe cannula 204 to help regulate a temperature of the antenna 208 and the ablation zone 210. Example cooling fluids include, but are not limited to, water, glycol, air, inert gases (e.g., helium), carbon dioxide, nitrogen, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

In some applications, the ablation probe 104 may include a stick region 214, alternately referred to as a “tissue-loc” region, provided on the probe cannula 204 at, near, or proximal to the antenna 208. The stick region 214 is designed to attain and maintain a temperature that accommodates adherence of a tissue region onto its surface. More specifically, the stick region 214 may operate as an anchoring element having a circulating agent or “coolant” (e.g., a gas delivered at or near its critical point; CO₂) that freezes the interface between the stick region 214 and the adjacent tissue, thereby sticking (maintaining, locking, etc.) the antenna 208 in place during operation. The coolant may be provided to the stick region 214 via the cooling tube 212 from the coolant source 107 (FIG. 1). Once a pre-determined low temperature is reached at the stick region 214, contact with adjacent tissue causes the tissue to adhere to the stick region 214, thereby resulting in attachment of the energy delivery device 204 to the tissue. During ablation, as the tissue warms, the antenna 208 remains secured to the tissue region due to tissue desiccation and charring. The stick region 214 may be made of any material able to attain and maintain a temperature such that contact with tissue induces adherence of the tissue onto the surface of the stick region 214. Example materials for the stick region 214 include, but are not limited to, a metal.

In some applications, the ablation probe 104 may further include a plug region 216 provided on the probe cannula 204 at or near the antenna 208. In at least one application, as depicted, the plug region 216 may be provided distal to the stick region 214 and otherwise interposing the stick region 214 and the antenna 208. The plug region 216 may be configured to prevent a reduction in temperature resulting from the cooled probe cannula 204 and the stick region 214 from affecting (e.g., reducing) the temperature within the antenna 208. In at least one other application, the plug region 216 may be provided distal to, or at least partially overlapping, the antenna 208, such that the coolant provided to the stick region 214 controls (reduces) the temperature of the tissue in contact with the antenna 208. Accordingly, the plug region 216 separates interior portions of the ablation probe 104 to prevent cooling or heating of a portion or portions of the device 104 while permitting cooling or heating of other portions. The plug region 216 may be made of an insulative material capable of being in contact with a material or region having a low temperature without having its temperature significantly reduced. Example insulative materials for the plug region 216 include, but are not limited to, a synthetic polymer (e.g., polystyrene, polyicynene, polyurethane, polyisocyanurate), aerogel, fiberglass, cork, or any combination thereof.

In some applications, the ablation probe 104 may further include a sharp stylet tip or “stylet” (not shown) positioned at the distal end of the antenna 208 and otherwise forming the distal end of the ablation probe 104. When included, the stylet is designed to facilitate percutaneous insertion of the ablation probe 104. The stylet may be made of a variety of rigid or hardened materials including, but not limited to, a hardened resin, a metal (e.g., titanium or an equivalent of titanium, stainless steel, etc.), a ceramic, a plastic or any combination thereof. In at least one application, the stylet may be braised to zirconia or an equivalent of zirconia. In such applications, the stylet may comprise an extension of a metal portion of the antenna 208 and may be electrically active. In some applications, the stylet may be formed from a dielectric material, such as ceramic or plastic, referenced above, such that the stylet is not an electrically active portion of the antenna 208.

In some embodiments, the ablation probe 104 may have a coaxial transmission line positioned within the antenna 208, and a coaxial transmission line connecting with the antenna 208. In some embodiments, the ablation probe 104 may comprise a triaxial microwave probe with optimized tuning capabilities. The ablation probe 104 may be the same as or similar to any of the energy delivery devices described in U.S. Pat. No. 11,638,607, entitled “ENERGY DELIVERY SYSTEMS AND USES THEREOF”, which issued on May 2, 2023, the contents of which are hereby incorporated by reference in their entirety herein.

Referring again to FIG. 1, the imaging system 116 is in communication with the controller 114 and comprises one or more imaging devices. Example imaging devices include, but are not limited to, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

In some applications, the imaging system 116 may be configured to monitor ablation procedures, such as monitoring the amount of ablation occurring within a particular tissue region(s) undergoing a thermal ablation procedure. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104.

The power supply 106 may be configured to supply the energy required to operate the system 100. The power supply 106 is also configured to supply energy to the ablation probes 104, such as microwave energy, radiofrequency energy, radiation, cryo energy, electroporation, high intensity focused ultrasound, or any combination thereof. In accordance with principles of the present disclosure, the power supply 106 supplies microwave energy to the ablation probes 104 for purposes of tissue ablation. More specifically, power may be supplied to the ablation probes 104, but the microwave energy is generated in a microwave generator and sent to the antenna 208. In some applications, the power supply 106 may include one or more energy generators configured to provide as much as 100 watts of microwave power of a frequency of from 915 MHz to 5.8 GHz, although the present invention is not so limited. The power splitter 108 may comprise a power distribution system operable to distribute the energy from the power supply 106 to the ablation probes 104. The power splitter 108 may be configured to provide varying energy levels to different regions of the ablation probes 104.

The temperature adjustment system 118 may be configured to use coolant systems (like the coolant source 107) and cooling fluids to help reduce undesired heating within and along the ablation probes 104. In particular, the temperature adjustment system 118 may include the coolant source 107 (FIG. 1) and the cooling tube 212 (FIG. 2) and may communicate with the handle 202 (FIG. 2) of each probe 104 to control conveyance of the cooling fluid into and out of the probe cannula 204 (FIG. 2), and thereby help regulate a temperature of the antenna 208 (FIG. 2) and the ablation zone 210 (FIG. 2). In some applications, the temperature adjustment system 118 may also be configured to communicate with the handle 202 to operate the stick region 214 and thereby attain and maintain a temperature that accommodates adherence of tissue onto its surface. For instance, in some embodiments, a user provides an input to an input interface, such as the GUI 120. Based on the user input, the temperature adjustment system 118 can control the coolant source 107 to provide coolant to the stick region 214, thereby adhering the stick region 214 to the adjacent tissue.

The temperature adjustment system 118 may also be configured to continuously or intermittently monitor the real-time temperature of the ablation probes 104. In such embodiments, the temperature adjustment system 118 may communicate with one or more temperature sensors (e.g., thermocouples) terminating at various points along the probe cannula 204 (FIG. 2) and/or the antenna 208 (FIG. 2) of the ablation probe 104. Consequently, localized temperature may be monitored at several points along the antenna 208 to estimate ablation status, cooling status, or safety checks. In some applications, monitoring the temperature at several points along the antenna 208 may help determine the geographical characteristics of the ablation zone 210 (FIG. 2), such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length. In some embodiments, the probe cannula 204 includes a first temperature sensor, a second temperature sensor, and a third temperature sensor. The first temperature sensor is placed at, or slightly proximal to, the antenna 208 to provide real-temperature measurements of the tissue being heated by the antenna 208. The second temperature sensor is placed at, or adjacent to, the stick region 214 to provide real-time temperature measurements of the tissue that is being cooled, and thus adhered to, the stick region 214. The third temperature sensor is proximal to the first and second temperature sensors along the cannula 204, such as at the point of entry into the skin, to provide real-time measurements of the patent's skin. The control system 102 can receive the temperature measurements from the first, second, and third sensors to control the coolant systems and cooling fluids from the temperature adjustment system 118 to the stick region 214 and/or other cooling systems of the energy delivery device 104.

The temperature adjustment system 118 may also be configured to monitor the temperature of a tissue region (e.g., tissue being treated, surrounding tissue). This may prove advantageous in helping to determine the status of the procedure (e.g., the end of the procedure). The temperature adjustment system 118 may communicate with the controller 114 to provide real-time temperature information to a user and display such measurements on the GUI 120. In at least one embodiment, based on the temperature data obtained by the temperature adjustment system 118, the controller 114 may be configured to autonomously adjust operation of the system 100 appropriately.

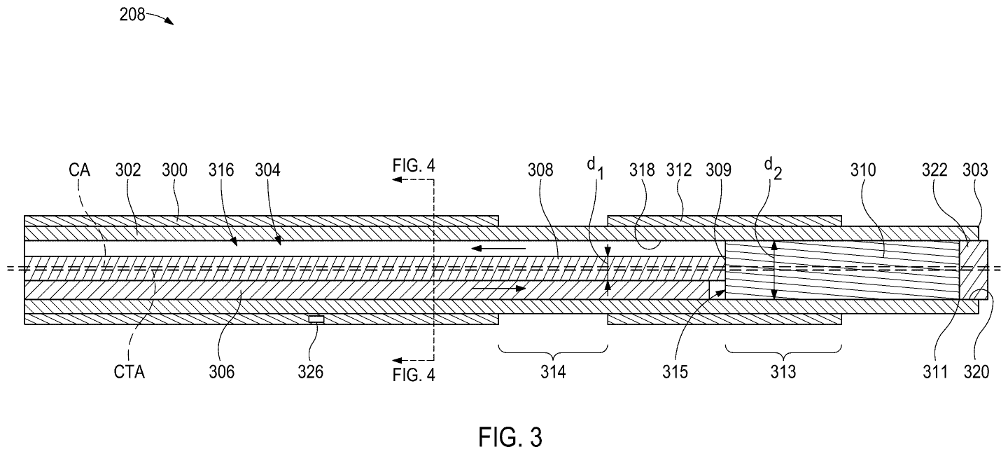

FIG. 3 is an enlarged, cross-sectional view of the antenna 208 of FIG. 2, according to at least one aspect of the present disclosure. As illustrated, the antenna 208 includes a coaxial cable arrangement for delivering energy to tissue. More specifically, the antenna 208 includes an outer conductor 300, a dielectric tube 302 extending within an interior of the outer conductor 300, an inner conductor 304 extending within an interior of the dielectric tube 302, and a coolant conduit or “tube” 306 also extending within the interior of the dielectric tube 302. The outer conductor 300 may be made of a conductive material that allows current to be transmitted along a length thereof. The outer conductor 300 may be made of a metal, for example, such as stainless steel, silver, copper, brass or aluminum, or alloys thereof. In some embodiments, the outer conductor 300 extends from the seal 216 (FIG. 2) and defines a longitudinal axis CTA. In at least one embodiment, the conductor 300 may comprise a 15 gauge hypotube.

The dielectric tube 302 extends through (within) the outer conductor 300 and may be concentric therewith. In some embodiments, the dielectric tube 302 is made of a polymer, such as polyamide, linear polyethylene (PE), polyethylene terephthalate (PET), polytetrafluoroethylene (PTFE) isotactic polypropylene (PP), or a polymer in the polyaryletherketone (PAEK) family, such as polyetheretherketone (PEEK), as examples.

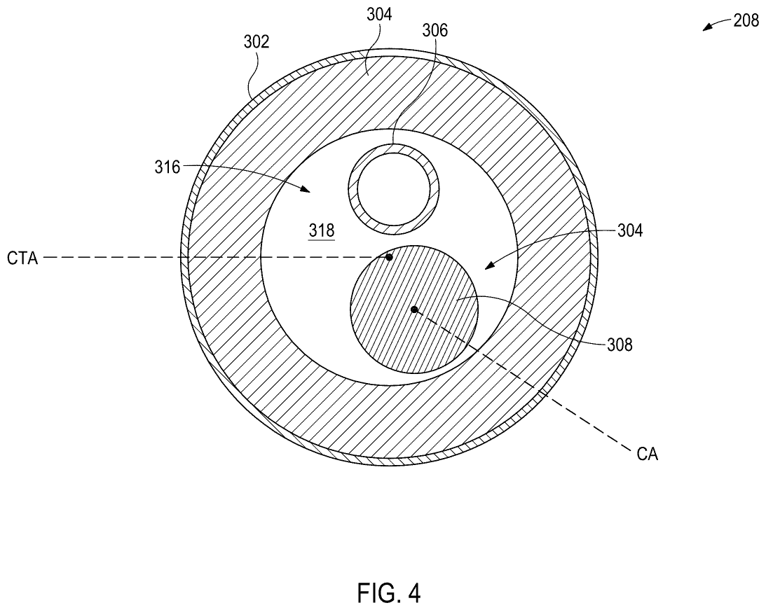

The inner conductor 304 extends through (within) the dielectric tube 302 and defines a longitudinal axis CA. In some embodiments, the inner conductor 304 may be concentrically positioned within the outer conductor 300 such that the longitudinal axis CA of the inner conductor 304 overlaps (is concentric with) the longitudinal axis CTA of the outer conductor 300. In other embodiments, however, the inner conductor 304 may be positioned such that the longitudinal axis CA is radially offset from or eccentric to the longitudinal axis CTA, as best shown in FIG. 4. In coaxial structures, radially offset (eccentric) conductors are typically avoided as the offset arrangement results in losses. However, in some embodiments, the inner conductor 304 may be intentionally radially offset (eccentric) to accommodate the coolant tube 306. Moreover, in some embodiments, the wall thickness of the dielectric tube 302 may be selected to limit the losses associated with radially offsetting the inner conductor 304 to within an acceptable threshold range. In some embodiments, the acceptable threshold range is 2-3%.

As illustrated, the inner conductor 304 may include a conductor shaft 308 (first portion) and a conductor load 310 (second portion) extending distally from the conductor shaft 308. The conductor shaft 308 defines a first diameter d₁ and the conductor load 310 defines a second diameter d₂ which is different (greater) than the first diameter d₁. In some embodiments, the conductor shaft 308 and the conductor load 310 are integrally constructed (formed), such as on a lathe from a piece of stock metal. In such embodiments, the conductor shaft 308 and the conductor load 310 may be made of the same material including, but not limited to, stainless steel, silver, copper, brass or aluminum, alloys thereof, or any combination thereof. In other embodiments, however, the conductor shaft 308 and the conductor load 310 may be separate component parts that are operatively coupled (e.g., welded, mechanically fastened, etc.). In such embodiments, the material of the conductor shaft 308 and conductor load 310 may be the same or different. As one example, the conductor shaft 308 may be made of copper and the conductor load 310 may be made of brass.

In some embodiments, a distal end 303 of the dielectric tube 302 extends beyond a distal end 311 of the conductor load 310 and thereby defines a distal recess 320. In some embodiments, the distal recess 320 may be filled with a plug 322 made of a thermoset polymer, such as polyurethane, an epoxy, vulcanized rubber, or vinyl ester, as examples. As mentioned above, in some applications, a stylet (not shown) may be positioned at the distal end of the antenna 208. In such embodiments, the stylet may be operatively coupled to a distal end of the plug 322, or may be an extension of the plug 322, to facilitate percutaneous insertion of the antenna 208 into a patient. In other embodiments, the plug 322 may be omitted and, alternatively, the stylet may be operatively coupled to the dielectric tube 302 and/or the conductor load 310 at the recess 320, such as with an adhesive, a press-fit engagement, or with a mechanical fastener, for example. As one example, the stylet may be a separate part that includes a trocar tip and a socket sized to fit over and attach to the distal end 303 of the dielectric tube 303, such with a press-fit connection. In some embodiments, the plug 322 may be omitted and the dielectric tube 302 may alternatively be thermoformed into the recess 322 to seal the distal end 303 of the dielectric tube 302. In such embodiments, the stylet may be coupled to the thermoformed end of the dielectric tube 302.

In some embodiments, the antenna 208 may comprise a dual slot antenna arrangement that includes a coupled, non-contacting distal short. In such embodiments, the dielectric tube 302 is uninterrupted and hermetically sealed. Moreover, in some embodiments, the conductor shaft 308 and conductor load 310 may be separated by a dielectric which acts as a high pass filter. In such embodiments, the presence of the dielectric creates an effective RF short despite being physically disconnected. In yet other embodiments, the dual slot antenna is terminated in a physically connected short that incorporates sealing structures to cap the end of the dielectric tube 302 using compression ferrules and/or adhesive bonds.

The inner conductor 304, and more specifically, the conductor shaft 308, may be in electrical communication with the power supply 106 (FIG. 1) to receive electrical energy therefrom. The received electrical energy is conveyed to the conductor load 310 and emitted therefrom to surrounding tissue. As discussed above, one factor contributing to an ellipsoid ablation shape is that the application of energy occurs along the length of the antenna 208 rather than from a point source. Providing the conductor 308 with a load point (i.e., the conductor load 310) with a longitudinal length tuned to match the dielectric properties of the surrounding tissue and probe materials contributes to the ability of the antenna 208 to generate the desired spherical ablation zone 210.

To further help generate a spherical ablation zone 210, the antenna 208 further includes a conductive sleeve 312 arranged about the dielectric tube 302 and distal to the outer conductor 300. As illustrated, the conductive sleeve 312 is longitudinally offset from the outer conductor 300 such that a gap 314 is defined between the choke tube 312 and the outer conductor 300. In some embodiments, the gap 314 may exhibit a length of about 5.5 mm, but could alternatively exhibit a length greater or less than 5.5 mm, without departing from the scope of the disclosure.

The conductive sleeve 312 may be made of the same material as the outer conductor 300, such as a conductive metal (e.g., stainless steel, silver, copper, brass or aluminum, or alloys thereof). In other embodiments, however, the conductive sleeve 312 may be made of a different material than the outer conductor 300. In some embodiments, the choke tube 312 and the outer conductor 300 may differ in length, but may have other similar dimensions. For example, the conductive sleeve 312 and the outer conductor 300 may each comprise 15 gauge hypotubes. In some embodiments, a portion 313 of the conductive sleeve 312 extends beyond (overlaps) a distal end 309 of the conductor shaft 308 and overlaps a proximal end 315 of the conductor load 310.

The conductive sleeve 312 may be configured to function as a parasitic element that may effectively “choke” return currents that may have a tendency to travel proximally along the outer conductor 300. More specifically, the gap 314 between the conductive sleeve 312 and the outer conductor 300 may be selected and otherwise sized to create destructive interference at, or near, the gap 314. The charge (current) induced on the inner surface of the conductive sleeve 312 couples at a phase that cancels charge (current) on the outer surface of the sleeve 312 at the gap 314. As discussed above, one factor contributing to an ellipsoid ablation shape is that current has a tendency to travel proximally (ride back) on the outer conductor 300 surface and is absorbed by tissue surrounding the probe proximal to the antenna 208. This may prove advantageous in helping to prevent back heating as current travels proximally on the unbalanced outer conductor 300 surface to be absorbed by tissue surrounding the proximal portions of the antenna 208. Accordingly, the conductive sleeve 312 helps to cancel or “choke” charge (current) riding proximally along the conductive sleeve 312, thus contributing to the ability of the antenna 208 to generate a spherical ablation zone 210.

The coolant tube 306 extends within the dielectric tube 302 alongside and parallel to the conductor 304. As illustrated, the coolant tube 306 extends through the antenna 208 to a point proximal to the conductor load 310 and located radially inward of the conductive sleeve 312. The coolant tube 306 is designed to receive and convey a coolant, such as those listed herein above (e.g., water, glycol, air, inert gases (e.g., helium), carbon dioxide, nitrogen, etc.) from a coolant source to control the temperature of the antenna 208 (via the Joule-Thompson effect) as the antenna 208 provides energy to the tissue of a patient.

In some embodiments, the coolant tube 306 may be fluidically coupled to the same coolant source as the cooling tube 212 (i.e., the coolant source 107 of FIG. 1). In other embodiments, however, the coolant tube 306 may be fluidically coupled to a separate coolant source than the cooling tube 212. The coolant tube 306 may be made of a polymer including, but not limited to, a polyamide, a polyimide (e.g., Kapton), linear polyethylene (PE), polyethylene terephthalate (PET), polytetrafluoroethylene (PTFE) isotactic polypropylene (PP), or a polymer in the polyaryletherketone (PAEK) family, such as polyetheretherketone (PEEK), or any combination thereof.

As shown in FIGS. 3 and 4, the dielectric tube 302 defines an interior or “channel” 316 sized and otherwise configured to receive the inner conductor 304 and the coolant tube 306. The remainder of the channel 316 that is unoccupied by the inner conductor 304 and the coolant tube 306 provides a return path 318 for the coolant supplied to the antenna 208 via the coolant tube 306. Accordingly, coolant can be circulated to the antenna 208 via the coolant tube 306 and away from the antenna 208 via the return path 318. In some embodiments, the return path 318 is fluidically coupled to a coolant sink (not shown) which receives the coolant from the return path 318. In some embodiments, the dielectric tube 302 is hermetically sealed to prevent the coolant provided to the antenna 208 from escaping into the patient. This may be particularly advantageous in embodiments where the coolant is a gas, such as CO₂.

In some embodiments, the control system 102 (FIG. 1) controls the flow of coolant from the coolant supply (e.g., the coolant supply 107 of FIG. 1) to the antenna 208 via the coolant tube 306. The rate of coolant flow may be predetermined. In some embodiments, the antenna 208 includes a temperature sensor 326 (FIG. 3) to measure the temperature of tissue in contact with, or adjacent to, the antenna 208. In some embodiments, the temperature sensor 326 is coupled to the outer conductor 300. In other embodiments, the temperature sensor 326 is coupled to the dielectric tube 302, such as in the gap 314 or to the choke tube 312. In such embodiments, the control system 102 can receive the sensed temperature from the temperature sensor 326 and control the rate of coolant flow based on the sensed temperature. For instance, the control system 102 can adjust (increase) the rate of coolant flow to the antenna 208 based on an upper threshold temperature being approached, reached, or exceeded.

When a user desires to ablate tissue surrounding the antenna 208, the user can provide an input to an input interface, such as the GUI 120. Based on the user input, the control system 102 may be programmed to control the power supply 106 (FIG. 1) to deliver energy to the antenna 208, as well as control the coolant supply to provide coolant to the antenna 208 via the coolant tube 306. In such embodiments, the control system 102 may be programmed to provide a continuous supply of the coolant to the antenna 208. In other embodiments, the control system 102 may be programmed to intermittently provide coolant to the antenna 208, such as at a predetermined frequency for predetermined amounts of time, or based on temperature measurements as sensed by the temperature sensor 326. The control system 102 may also be programmed to provide coolant to the stick region 214 (FIG. 2) and the antenna 208 via the coolant tube 316 in an overlapping timeframe such that the stick region 214 and the antenna 208 co-operatively control the temperature of the antenna 208 and, thus, the shape of the ablation zone 210.

In some embodiments, coolant supplied (circulated) to the antenna 208 via the coolant tube 306 serves to prevent tissue charring and to aid the conductive sleeve 312 in performing its intended function. The coolant provided to the antenna 208 via the coolant tube 306 removes or reduces resistive heating and inefficiencies that contribute to proximal heating along the energy delivery device 104. As discussed above, one factor contributing to an ellipsoid ablation shape is that the dielectric properties of tissue change by more than an order of magnitude as the tissue temperature changes. This creates an inefficient, non-optimized environment for the antenna 208 to operate, thereby causing tissue desiccation and charring, which creates back-heating along the length of the energy delivery device and contributes to the ablation zone having an ellipsoid shape. Providing coolant to the antenna 208 via the coolant tube 306 minimizes the change in dielectric properties of the tissue which prevents, or reduces, the chance of tissue desiccation and/or charring and back-heating along the length of the energy delivery device 104, thus contributing to the ability of the antenna 208 to generate the desired spherical ablation zone 210.

The embodiments of the antenna 208 described herein provide a clinician with the ability to create spherical (or at least substantially spherical) shaped ablation zones 210 (FIG. 2) as opposed to ellipsoid (teardrop) shaped ablation zones. Notably, the ability of the antenna 208 to generate a spherical ablation zone 210 reduces the importance of the approach angle to a target tissue as the spherical ablation zone can cover the same target tissue regardless of approach angle. This provides the clinician with more freedom (options) for how to approach the target tissue. This is particularly important as it allows clinicians to avoid critical structures (arteries, organs, tumors, etc.) or place the antenna 208 along an advantageous anatomical or imaging path without concern for how the ablation volume will overlay the target tissue.

Initial prototyping of the energy delivery device 104 with the antenna 208 created nearly spherical ablation zones (3.5cm×3.6 cm in liver at 80 W for 5 minutes, and 4.4 cm×4.5 cm at 120 W for 6 minutes). Furthermore, the prototyped energy delivery device 104 demonstrated the ability to lock onto tissue using the stick region 214, which satisfied an unmet need of a 15 gauge or smaller ablation device that sticks (locks) onto tissue and safety generates spherical ablations with microwave energy. Other advantages associated with generating a spherical ablation zone as opposed to an ellipsoid ablation zone will be readily apparent to a person having ordinary skill in the art.

Embodiments disclosed herein include:
A. An energy delivery device comprising a cannula extendable through skin of a patient and an antenna extending from the cannula and operable to deliver energy to tissue of the patient, wherein the antenna includes an outer conductor, a dielectric tube extending within the outer conductor, an inner conductor extending within the dielectric tube, and a coolant tube extending within the dielectric tube to convey a coolant to the antenna.
B. A system comprising an energy delivery device including an antenna operable to deliver energy to tissue of a patient and a controller. The antenna includes an outer conductor, a dielectric tube extending within the outer conductor, an inner conductor extending within the dielectric tube, and a coolant tube extending within the dielectric tube to convey a coolant to the antenna. The controller is operable configured to control a flow of coolant from a coolant supply through the coolant tube and to the antenna.
C. A method comprising advancing an antenna of an energy delivery device toward the tissue, the antenna including an outer conductor, a dielectric tube extending within the outer conductor, an inner conductor extending within the dielectric tube, a conductive sleeve arranged about the dielectric tube and axially offset from the outer conductor such that an axial gap is defined therebetween. The method further comprises conveying a current distally to the antenna via the inner conductor, preventing a return current from travelling proximally on the outer conductor with the conductive sleeve and thereby preventing back heating of the tissue along the outer conductor, and generating a substantially spherical ablation zone in the tissue by preventing the return current from travelling proximally on the outer conductor.

Each of embodiments A, B, and C may have one or more of the following additional elements in any combination: Element 1: wherein the outer conductor defines a first longitudinal axis and the inner conductor defines a second longitudinal axis eccentric to the first longitudinal axis. Element 2: wherein the antenna further includes a conductive sleeve arranged about the dielectric tube and longitudinally offset from the outer conductor such that a gap is defined therebetween. Element 3: wherein the conductive sleeve prevents back heating along the outer conductor. Element 4: wherein the inner conductor comprises a conductor shaft that exhibits a first diameter and a conductor load extending from the conductor shaft and exhibiting a second diameter greater than the first diameter. Element 5: wherein the conductor shaft is made of a first material and the conductor load is made of a second material different than the first material. Element 6: wherein the antenna further includes a conductive sleeve arranged about the dielectric tube and longitudinally offset from the outer conductor such that a gap is defined therebetween, and a portion of the conductive sleeve overlaps a proximal end of the conductor load. Element 7: wherein the controller includes an input interface and is further operable to receive, at the input interface, a user input and control, from a power supply, energy delivery to the antenna based on the user input, wherein controlling the flow of coolant through the coolant tube to the antenna is based on the user input. Element 8: further comprising a temperature sensor in communication with the controller and operable to sense a temperature of the tissue, wherein controlling the flow of coolant through the coolant tube to the antenna is based on the temperature of the tissue. Element 9: wherein the outer conductor defines a first longitudinal axis and the inner conductor defines a second longitudinal axis eccentric to the first longitudinal axis. Element 10: wherein the antenna further includes a conductive sleeve arranged about the dielectric tube and longitudinally offset from the outer conductor such that a gap is defined therebetween. Element 11: wherein the conductive sleeve prevents back heating along the outer conductor. Element 12: wherein the inner conductor comprises a conductor shaft that exhibits a first diameter and a conductor load extending from the conductor shaft and exhibiting a second diameter greater than the first diameter. Element 13: wherein the antenna further includes a conductive sleeve arranged about the dielectric tube and offset from the outer conductor such that a gap is defined therebetween, and a portion of the conductive sleeve extends beyond a distal end of the conductor shaft. Element 14: wherein the antenna further includes a coolant tube extending within the dielectric tube, the method further comprising conveying a coolant from a coolant supply through the coolant tube and to the antenna, discharging the coolant from the coolant tube at a location proximal to a distal end of the antenna, and circulating the coolant proximally within a return path defined within the dielectric tube, wherein circulating the coolant to and from the antenna further helps generate the substantially spherical ablation zone in the tissue. Element 15: further comprising receiving, at an input interface, a user input, wherein conveying the current distally to the antenna via the inner conductor and conveying the coolant from the coolant supply through the coolant tube and to the antenna are based on receiving the user input. Element 16: further comprising sensing, with a temperature sensor, a temperature of the tissue, wherein conveying the coolant from the coolant supply through the coolant tube and to the antenna is based on the sensed temperature. Element 17: wherein the outer conductor defines a first longitudinal axis and the inner conductor defines a second longitudinal axis eccentric to the first longitudinal axis.

By way of non-limiting example, exemplary combinations applicable to A, B, and C include: Element 1 with Element 2; Element 1 with Elements 2 and 3; Element 1 with Element 4; Element 1 with Elements 4 and 5; Element 1 with Elements 4 and 6; Element 2 with Element 3; Element 2 with Element 4; Element 2 with Elements 4 and 5; Element 2 with Elements 4 and 6; Elements 2 and 3 with Element 4;Elements 2 and 3 with Elements 4 and 5; Elements 2 and 3 with Elements 4 and 6; Element 4 with Element 5; Element 4 with Element 6; Element 1 with at least two of Elements 2-6; Element 2 with at least two of Elements 1 and 3-6; Element 3 with at least two of Element 1, 2, and 4-6; Element 4 with at least two of Elements 1-3, 5, and 6; Element 5 with at least two of Elements 1-4 and 6; Element 6 with at least two of Elements 1-5; Element 8 with Element 9; Element 8 with Element 10; Element 8 with Element 11; Element 8 with Elements 11 and 12; Element 8 with Element 13; Element 8 with Elements 13 and 14; Element 9 with Element 10; Element 9 with Element 11; Element 9 with Elements 11 and 12; Element 9 with Element 13; Element 9 with Elements 13 and 14; Element 10 with Element 11; Element 10 with Elements 11 and 12; Element 10 with Element 13; Element 10 with Elements 13 and 14; Element 11 with Element 12; Element 11 with Element 13; Element 11 with Elements 13 and 14; Elements 11 and 12 with Element 13; Elements 11 and 12 with Elements 13 and 14; Element 13 with Element 14; Element 8 with at least two of Elements 9-14; Element 9 with at least two of Elements 8 and 10-14; Element 10 with at least two of Elements 8, 9, and 11-14; Element 11 with at least two of Elements 8-10 and 12-14; Element 12 with at least two of Elements 8-11, 13, and 14; Element 13 with at least two of Elements 8-12 and 14; Element 14 with at least two of Elements 8-13; Element 15 with Element 16; Element 15 with Element 17; and Element 15 with Element 18; Element 15 with Elements 16 and 17; Element 15 with Elements 16 and 18; Element 15 with Elements 17 and 18; and Element 15 with Elements 15-18.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. An energy delivery device, comprising:
a cannula extendable through skin of a patient; and
an antenna extending from the cannula and operable to deliver energy to tissue of the patient, wherein the antenna includes:
an outer conductor;
a dielectric tube extending within the outer conductor;
an inner conductor extending within the dielectric tube; and
a coolant tube extending within the dielectric tube to convey a coolant therethrough.

2. The energy delivery device of claim 1, wherein the outer conductor defines a first longitudinal axis and the inner conductor defines a second longitudinal axis eccentric to the first longitudinal axis.

3. The energy delivery device of claim 1, wherein the antenna further includes a conductive sleeve arranged about the dielectric tube and longitudinally offset from the outer conductor such that a gap is defined therebetween.

4. The energy delivery device of claim 3, wherein the conductive sleeve prevents back heating along the outer conductor.

5. The energy delivery device of claim 1, wherein the inner conductor comprises:
a conductor shaft that exhibits a first diameter; and
a conductor load extending from the conductor shaft and exhibiting a second diameter greater than the first diameter.

6. The energy delivery device of claim 5, wherein the conductor shaft is made of a first material and the conductor load is made of a second material different than the first material.

7. The energy delivery device of claim 5, wherein the antenna further includes a conductive sleeve arranged about the dielectric tube and longitudinally offset from the outer conductor such that a gap is defined therebetween, and a portion of the conductive sleeve overlaps a proximal end of the conductor load.

8. A system, comprising:
an energy delivery device including an antenna operable to deliver energy to tissue of a patient, the antenna including:
an outer conductor;
a dielectric tube extending within the outer conductor;
an inner conductor extending within the dielectric tube; and
a coolant tube extending within the dielectric tube to convey a coolant to the antenna; and
a controller operable to control a flow of coolant from a coolant supply through the coolant tube and to the antenna.

9. The system of claim 8, wherein the controller includes an input interface and is further operable to:
receive, at the input interface, a user input; and
control, from a power supply, energy delivery to the antenna based on the user input,
wherein controlling the flow of coolant through the coolant tube to the antenna is based on the user input.

10. The system of claim 8, further comprising a temperature sensor in communication with the controller and operable to sense a temperature of the tissue, wherein controlling the flow of coolant through the coolant tube to the antenna is based on the temperature of the tissue.

11. The system of claim 8, wherein the outer conductor defines a first longitudinal axis and the inner conductor defines a second longitudinal axis eccentric to the first longitudinal axis.

12. The system of claim 8, wherein the antenna further includes a conductive sleeve arranged about the dielectric tube and longitudinally offset from the outer conductor such that a gap is defined therebetween.

13. The system of claim 12, wherein the conductive sleeve prevents back heating along the outer conductor.

14. The system of claim 8, wherein the inner conductor comprises:
a conductor shaft that exhibits a first diameter; and
a conductor load extending from the conductor shaft and exhibiting a second diameter greater than the first diameter.

15. The system of claim 14, wherein the antenna further includes a conductive sleeve arranged about the dielectric tube and offset from the outer conductor such that a gap is defined therebetween, and a portion of the conductive sleeve extends beyond a distal end of the conductor shaft.

16. A method, comprising:
advancing an antenna of an energy delivery device toward the tissue, the antenna including:
an outer conductor;
a dielectric tube extending within the outer conductor;
an inner conductor extending within the dielectric tube; and
a conductive sleeve arranged about the dielectric tube and offset from the outer conductor such that an axial gap is defined therebetween;
conveying a current distally to the antenna via the inner conductor;
preventing a return current from travelling proximally on the outer conductor with the conductive sleeve and thereby preventing back heating of the tissue along the outer conductor; and
generating a substantially spherical ablation zone in the tissue by preventing the return current from travelling proximally on the outer conductor.

17. The method of claim 16, wherein the antenna further includes a coolant tube extending within the dielectric tube, the method further comprising:
conveying a coolant from a coolant supply through the coolant tube and to the antenna;
discharging the coolant from the coolant tube at a location proximal to a distal end of the antenna; and
circulating the coolant proximally within a return path defined within the dielectric tube,
wherein circulating the coolant to and from the antenna further helps generate the substantially spherical ablation zone in the tissue.

18. The method of claim 17, further comprising receiving, at an input interface, a user input, wherein conveying the current distally to the antenna via the inner conductor and conveying the coolant from the coolant supply through the coolant tube and to the antenna are based on receiving the user input.

19. The method of claim 17, further comprising sensing, with a temperature sensor, a temperature of the tissue, wherein conveying the coolant from the coolant supply through the coolant tube and to the antenna is based on the sensed temperature.

20. The method of claim 17, wherein the outer conductor defines a first longitudinal axis and the inner conductor defines a second longitudinal axis eccentric to the first longitudinal axis.
